(19) United States
(12) Patent Application Publication
(10) Pub. No.: US 2026/0306365 A1
(43) Pub. Date: Oct. 8, 2026

(54) NASOGASTRIC DEVICE AND USES THEREOF

(71) Applicants: University of Connecticut, Farmington, CT (US); Connecticut Children's Medical Center, Hartford, CT (US)

(72) Inventors: Christine FINCK, Glastonbury, CT (US); Liisa KUHN, West Hartford, CT (US); Ken PREMO, Windsor Locks, CT (US)

(21) Appl. No.: 19/482,556

(22) PCT Filed: May 13, 2024
(86) PCT No.: PCT/US2024/029175

§ 371 Date: Nov. 7, 2025

Related U.S. Application Data
(60) Provisional application No. 63/465,549, filed on May 11, 2023.

(51) Int. Cl. A61J 15/00

(52) U.S. Cl. CPC A61J 15/0003; A61J 15/0049; A61J 15/0073; A61J 15/0088

(57) ABSTRACT

Disclosed herein is a nasogastric device comprising a multi-lumen tubular conduit having a proximal end and a distal end with a plurality of conduits arranged therebetween; a plurality of inlet ports located at the proximal end in fluid communication with the plurality of conduits; where the plurality of conduits are arranged such that at least one conduit of the plurality of conduits is located with a wall of another conduit; where at least one conduit of the plurality of conduits is in fluid communication with an inflatable balloon; and where at least one conduit of the plurality of conduits is in fluid communication with a drug delivery inlet port.

### CROSS-REFERENCE TO RELATED APPLICATIONS

This disclosure claims priority to U.S. Provisional Application No. 63/465,549, filed on May 11, 2023, the entire contents of which are incorporated herein in their entirety.

### BACKGROUND

This disclosure relates to nasogastric devices for treatment of esophageal narrowing and other esophageal conditions including enteral nutrition.

Numerous medical conditions affecting both adults and children require the replacement or repair of a section of the esophagus. These conditions include esophageal atresia, caustic ingestions, and esophageal carcinomas. Esophageal atresia (“EA”) is a rare congenital abnormality where a baby is born with an incomplete esophagus and the inability for food to travel from the mouth to the stomach. It may be estimated that esophageal atresia occurs in 1 of every 2,500-3,500 live births. Further each year several thousand children worldwide are born with one of the four types of esophageal atresia.

In the United States, an estimated 5,000-15,000 caustic ingestions occur per year. The ingestion of these chemicals can be a devastating injury depending on the amount of esophageal tissue that becomes necrotic. Accidental ingestions of button or coin shaped batteries is around 2,500/year and rising. The larger 2V batteries result in a rapid circumferential injury that must be treated quickly to avoid death. Esophageal carcinoma is the sixth most common cause of cancer death in the world each year. Furthermore, the five-year survival rate ranges from 15-20%. In addition to radiation treatment, surgery is typically the most successful treatment for esophageal carcinoma as the cancerous segment is removed.

Current surgical options available for the treatment of EA, esophageal carcinoma, button batteries or caustic ingestions focus upon either the replacement of the esophageal tissue with other autologous tissue like gastric pull-up or colon interposition, or mechanically stretching the current esophageal tissue to bridge the gap, such as the Foker procedure. The Foker procedure is performed by placing sutures on the small pieces of esophagus that are present and stretching them over a period of time, followed by connection of the two ends. Although this procedure results in the use of esophageal tissue to replace this gap, there are significant complications. These complications include anastomotic leakage and esophageal strictures. Such an approach also requires at least two separate surgical interventions.

Colon or small intestine can be utilized to bridge the gap; however, since the structure and function of these tissues is not the same as the esophagus, the approach can associate with complications including reflux, strictures or even subsequent replacement of the segment due to significant dilation. A gastric pull-up procedure can also be performed that tubularizes the patient's stomach and brings it up into the patient's thoracic cavity. Complications seen with this type of procedure are similar to that of the other repair options mentioned above. Furthermore, the FDA recommends no more than 1 anesthesia routine before the age of 5.

It is therefore desirable to develop devices and methods that facilitate treatment of esophageal narrowing and other esophageal conditions.

### SUMMARY

Disclosed herein is a nasogastric device comprising a multi-lumen tubular conduit having a proximal end and a distal end with a plurality of conduits arranged therebetween; a plurality of inlet ports located at the proximal end in fluid communication with the plurality of conduits; where the plurality of conduits are arranged such that at least one conduit of the plurality of conduits is located with a wall of another conduit; where at least one conduit of the plurality of conduits is in fluid communication with an inflatable balloon; and where at least one conduit of the plurality of conduits is in fluid communication with a drug delivery inlet port.

Disclosed herein too is a nasogastric device, the device comprising a multi-lumen tubular conduit having a proximal end and a distal end with a plurality of conduits arranged therebetween; the proximal end having a plurality of inlet ports and the distal end having a plurality of outlet ports; where a first conduit of the plurality of conduits is in communication with a first inlet port and a first outlet port; where the first inlet port is operative deliver a biologically active agent to the first outlet port; and where a second conduit of the plurality of conduits is in communication with a second inlet port and a second outlet port; where the second inlet port is operative to pressurize an inflatable balloon that lies closer to the distal end than the first outlet port; where the second conduit lies inside the first conduit or lies adjacent to the first conduit.

Disclosed herein too is a method of treating an esophageal stricture with a nasogastric device, the method comprising threading the nasogastric device through a nose of a living being into an esophagus; and inflating an inflatable balloon in the nasogastric device to expand a stricture in the esophagus; where the nasogastric device comprises a multi-lumen tubular conduit having a proximal end and a distal end with a plurality of conduits arranged therebetween; the proximal end having a plurality of inlet ports and the distal end having a plurality of outlet ports; where a first conduit of the plurality of conduits is in communication with a first inlet port and a first outlet port; where the first inlet port is operative deliver a biologically active agent to the first outlet port; and where a second conduit of the plurality of conduits is in communication with a second inlet port and a second outlet port; where the second inlet port is operative to pressurize the inflatable balloon that lies closer to the distal end that the first outlet port; where the second conduit lies inside the first conduit or lies adjacent to the first conduit.

### BRIEF DESCRIPTION OF THE FIGURES

FIG. 1 is a depiction of an exemplary nasogastric device;

FIG. 2A is a depiction of a cross-section of the multi-lumen tubular device that contains multiple conduits;

FIG. 2B is a side view of the multi-lumen tubular device of the FIG. 2A;

FIG. 2C is a side view of a cross-section of a sheath where the conduits are arranged to be side-by-side;

FIG. 3 is a depiction of the inflatable balloon and stricture sensors; and

FIGS. 4A-4C include depictions of the nasogastric device being used to locally deliver a drug and collect or wash out the drug afterwards.

### DETAILED DESCRIPTION

Disclosed herein is a nasogastric device that may be used for expanding esophageal strictures or for treating other esophageal damage in the body of a living being. The nasogastric device includes comprises a monolithic multi-lumen tubular device that includes a plurality of conduits that can be threaded through the nose of a patient, down the esophagus and into the stomach to amongst other things expand strictures and to deliver drugs to a damaged site in the esophagus and nutrients to the patient. The multi-lumen tubular device comprises a plurality of conduits at least one of which surrounds all the other conduits or wherein all conduits lie side-by-side. Each conduit is fitted with an outlet port. At least one conduit of the plurality of conduits is in fluid communication with an inflatable balloon that comprises an inner inflatable sleeve and an outer elastomeric sleeve. The inflatable sleeve and outer elastomeric sleeve surround the plurality of conduits (but not the drug delivery ports) and are affixed to the multi-tubular device by radio-opaque clasps at both ends. Disposed between the inflatable sleeve and the outer elastomeric sleeve are sensors that can provide caretakers or medical personnel with information about the development of strictures in the esophagus.

Disclosed herein too is a method of using the nasogastric feeding tube to expand esophageal strictures. The nasogastric feeding tube may be introduced into an esophageal cavity via a nasal cavity to perform several functions such as, for example, expanding an esophageal stricture or a region proximate to a stricture (hereinafter the “stricture”), temporarily reinforcing the esophagus during repair or recovery, delivering drugs to the stricture or to damaged tissue and removing fluids and tissue from the stricture for analysis.

When esophageal strictures occur in patients undergoing esophageal surgery, dilation is used. This is a procedure that typically uses anesthesia and is not cured with a single dilation, rather needs repetitive dilations over months. Accordingly, weekly to monthly dilations are performed using an endoscope and pneumatic dilator. In a separate procedure under anesthesia, a gastrostomy feeding tube (“G-Tube”) is surgically placed in the stomach and brought out through the abdominal wall to allow for enteral feeding to provide nutrition until the esophagus can be repaired. In some refractory cases of esophageal stricture, topical application of medicine such as mitomycin C has been described with some benefit. However, this method also uses endoscopy with directed delivery of the medicine.

In severe cases, such as seen in esophageal cancer, esophageal stents are placed which also requires endoscopy and anesthesia. The known common complication from stenting is migration of the stent requiring endoscopic removal and replacement. Drugs are currently delivered orally by swallowing a suspension like steroids, which treats the entire esophagus and is not targeted to the specific site of concern.

As noted above, the nasogastric device includes a monolithic multi-lumen tubular conduit (hereinafter “multi-lumen tubular conduit” or “multi-lumen conduit”) that includes a plurality of conduits. In an embodiment, at least one conduit (a first conduit) is in fluid communication with a drug delivery inlet port (a first port) (i.e., transport suspensions or solutions of materials, such as therapeutic molecules (hereinafter therapeutic materials) that opens into a space exterior and proximal to the inflatable balloon) while an adjacent conduit (a second conduit) is in fluid communication with a balloon dilation and sensing inlet port (a second port). The second conduit is configured to inflate an expandable balloon (hereinafter an inflatable balloon) to expand strictures that may occur in the esophagus. An optional third conduit within the multi-tubular conduit is in communication with an aspiration port (a third inlet port) and is configured to terminate inside the esophagus. This third conduit can facilitate aspiration (removal of excess therapeutics that are not absorbed by the body) and can further be configured to extract tissue from the esophagus or stomach to conduct an analysis of the local environment for determining a course of treatment. An optional fourth conduit within the multi-tubular conduit is in fluid communication with a feeding tube port (a fourth inlet port) and is configured to deliver nutrients to the patient. It may be the longest of the respective conduits and terminates inside the stomach. Each of the conduits is of increasing length, with the fourth conduit being longer than the third conduit, which is longer than the second conduit. The first conduit is generally the shortest conduit.

In an embodiment, in addition to the inlet ports, there may be electrical and optical communications that facilitate sensing operations in the inflatable balloon. The electrical and optical communications can be in communication with microprocessors near the proximal end of the nasogastric device. These electrical wires or fiber optic cables may be enclosed within one of the conduits and perhaps in its own conduit.

### The Device and Inlet Ports

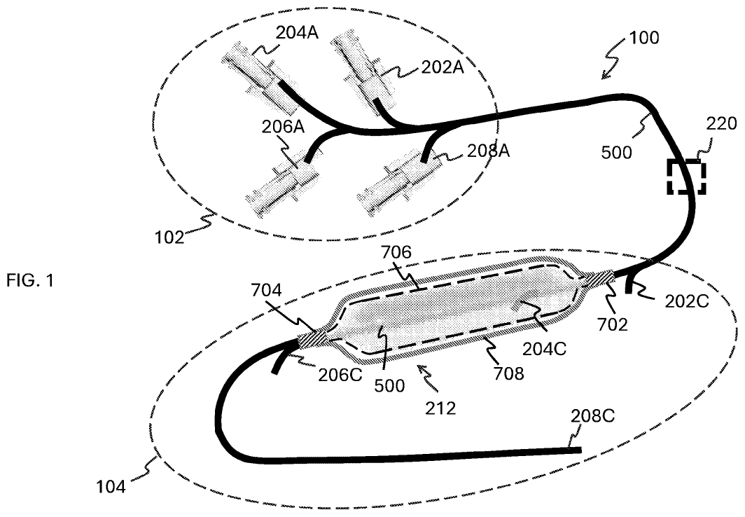

With reference now to the FIG. 1, the nasogastric device 100 comprises a proximal end 102 (the end of the device that is closest to a care giver controlling the device at the time the care-giver is using the device) and a distal end 104 (the end opposite to the distal end that is typically farthest away from the care giver controlling the device) with a multi-lumen tubular conduit 500 disposed therebetween. In an embodiment, the proximal end 102 lies upstream of the distal end 104. In an embodiment, the nasogastric device can function in the manner of a recycle loop for certain operations. For example, it can transport some materials from the proximal end to the distal end during use in the esophagus, while sequentially or simultaneously transporting the same or other materials from the distal end to the proximal end via the same conduit or another conduit.

The multi-lumen tubular conduit 500 comprises a plurality of conduits each of which has an inlet port at the proximal end 102 with individual outlet ports located further downstream of the inlet port. Each inlet port may be fitted with an inlet device that is designed to complement the function that the respective conduit is designated to handle. Each inlet device is detachable and can be reattached when desired. The inlet devices can also be interchanged if desired. The respective outlet ports are located at different regions of the multi-lumen tubular conduit depending upon the functionality of the particular conduit. This will be discussed in detail later. An inflatable balloon lies downstream of the proximal end and is in fluid communication with at least one of the conduits in the multi-lumen tubular conduit. The inflatable balloon can be used to expand an existing stricture or alternatively, to provide information about a developing stricture based on the pressure exerted by the stricture on the balloon.

Located at the proximal end 102 are a plurality of inlet ports—a first inlet port 202A configured to handle drug delivery (i.e., to transport suspensions or solutions of materials, such as therapeutic molecules into a space exterior to said inflatable balloon) to the site in the esophagus that is being treated, a second inlet port 204A (also sometimes referred to as a balloon dilation and sensing port) that is in fluid communication with the inflatable balloon 212 (that is located at the distal end 104), a third inlet port 206A (also sometimes referred to as an aspiration port) that facilitates aspiration (removal) of excess therapeutics that are not absorbed by the body and that is configured to extract fluid or tissue from the esophagus or stomach for sampling of the local environment, and a fourth inlet port 208A (sometimes referred to as a feeding tube port) configured to deliver nutrients to the stomach of the patient.

Each inlet port—the first inlet port 202A, the second inlet port 204A, the third inlet port 206A and the fourth inlet port 208A may be fitted with an appropriate device for injecting a fluid from the proximal end 102 towards the distal end 104 or for extracting a fluid or tissue from the distal end 104 and transporting it to the proximal end 102 of the nasogastric device 100. It is therefore to be recognized that each inlet port may also be used as an outlet port or vice versa. In other words, each inlet port may be used for injecting or extracting fluids (e.g., air or water), foods and tissue, while each outlet port may be used for introducing and extracting fluids, food and tissue.

In an embodiment, the first inlet port 202A is in communication with a pump (not shown) that delivers drugs and nutrients to the esophagus. In an embodiment, the pump affixed to the drug delivery port may be an injection pump. The second inlet port 204A may be in communication with a hand controlled pump such as, for example, a bulb or squeeze bulb (not shown) that can be squeezed by hand or electronically to introduce a fluid into the inflatable balloon 212.

The third inlet port 206A is configured to facilitate fluid aspiration or to remove tissue from the esophagus for observation and study. The third inlet port 206A is in communication with a reversible pump (not shown) that can be used to extract excess therapeutic materials from an esophageal region that lies downstream of the inflatable balloon. This reversible pump is generally used in a suction mode (where it is used to suck up therapeutic materials that lie downstream of the inflatable balloon).

The fourth inlet port 208A is in communication with a feeding pump (not shown) that may be used to deliver nutrients to the esophagus. The feeding pump in communication with the fourth inlet port may also be an injection pump.

The pumps connected to the respective inlet ports can be programmed to deliver the desired medication or nutrients in precise amounts over a set period of time. These pumps may be in electrical communication with a microprocessor that receives information from the nasogastric device 100 and adjusts inputs accordingly.

Located at the distal end 104 of the nasogastric device 100 are a plurality of outlet ports 202C, 204C, 206C and 208C each of which are in fluid communication or in electrical or light communication with at least one of the inlet ports 202A, 204A, 206A and 208A respectively or another port for the stricture sensor. Inlet ports 202A, 204A, 206A and 208A are in communication with conduits 202B, 204B, 206B and 208B respectively, which in turn communicate with outlet ports 202C, 204C, 206C and 208C respectively. The conduits along with the corresponding inlet and outlet ports are discussed in detail below.

### The Conduits

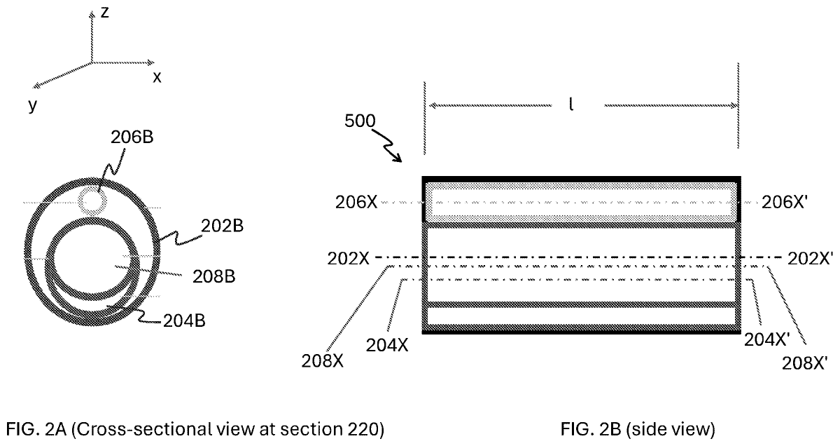

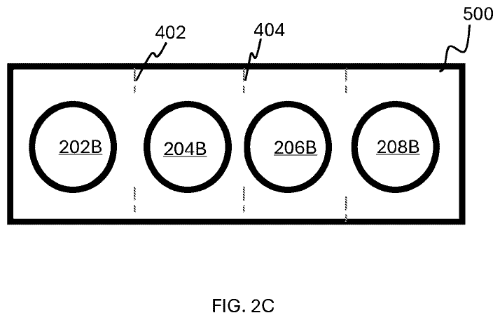

FIG. 2A is a depiction of one embodiment of an exemplary cross-section of the multi-lumen tubular conduit 500 taken at region 220 of FIG. 1. FIG. 2B is a side view of the multi-lumen tubular conduit 500 of length “l”. From the FIG. 2A and FIG. 1, it may be seen that the multi-lumen tubular conduit 500 comprises a plurality of conduits—the first conduit 202B which facilitates drug delivery from first inlet port 202A to first outlet port 202C; second conduit 204B which facilitates the transportation of an inflating fluid to inflate the balloon 212 from second inlet port 204A to the second outlet port 204C; the third conduit 206B which facilitates aspiration and tissue removal and which is in fluid communication with third inlet port 206A and third outlet port 206C; and a fourth conduit 208B which is operative to feed nutrients to a patient undergoing treatment and which is in communication with fourth inlet port 208A and fourth outlet port 208C

From the FIG. 2A it may be seen that the multi-lumen tubular conduit 500 has a multi-lumen design and comprises the first conduit 202B that encompasses each of the other conduits namely—second conduit 204B, third conduit 206B and fourth conduit 208B. The conduits 204B, 206B and 208B lie within the walls of conduit 202B. In an embodiment, conduit 204B always lies within the wall of the conduit 202B (since it opens into the inflatable balloon 212), while conduits 206B and 208B lie within the walls of conduit 202B but protrude outside the conduit 202B only at those locations where they have outlet ports.

A multi-lumen design refers to a structure or device that has multiple lumens or channels within it. The multi-lumen design means that the device has several separate pathways or channels for the flow of fluids, gases, or other materials. In an exemplary embodiment, the conduits are coextruded together and cannot be separated from each other without destroying the structure of the multi-lumen tubular conduit and the conduits within the multi-lumen tubular conduit.

Each conduit 202B, 204B, 206B and 208B has a longitudinal axis that is parallel to the longitudinal axis of the multi-lumen tubular conduit 500. The longitudinal axis of each conduit is the central axis that traverses the entire length of that particular conduit. The longitudinal axis of the multi-lumen tubular conduit 500 (202X-200X′) (which is also the longitudinal axis of the conduit 202B) lies parallel to the longitudinal axis of the conduits 206B (206X-206X′), 204B (204X-204X′) and 208B (208X-208X′). It may be seen that each of the longitudinal axes lie in a single plane when viewed in one direction (the z-direction and/or the y-direction) but lie in a different plane when viewed in the x-direction. It can be seen that the respective conduits 204B, 206B and 208B are mounted eccentrically with respect to the conduit 202B that encompasses all of them. The conduits are therefore able to transmit fluids or electrical or light signals independently of each other.

In an embodiment, the conduit 204B (which is operative to inflate the balloon 212) is located closest to an inner wall of the conduit 202B. From the FIG. 2A it may be seen that each conduit has an approximately circular cross-sectional area, where one conduit 202B encompasses all the other conduits—namely 204B, 206B and 208B. The conduit 202B therefore functions as the multi-lumen tubular conduit 500 for all of the other conduits—204B, 206B and 208B. The conduits 204B and 208B share an outer wall. The conduit 206B lies within conduit 202B but lies outside the outer wall of conduits 204B and 208B.

FIG. 2C depicts another cross-sectional view of the multi-lumen tubular conduit 500 that encompasses all of the conduits. In this view, the multi-lumen tubular conduit 500 has a rectangular or square cross-sectional area that encompasses each of the conduits 202B, 204B, 206B and 208B. In this embodiment, the conduits are arranged to be side-by-side. The multi-lumen tubular conduit 500 has perforation lines 402, 404, and so on, located on an outer periphery of the multi-lumen tubular conduit 500 between the conduits 202B, 204B, 206B and 208B. These perforation lines facilitate a separation of the ends of the conduits from each other at the proximal and distal ends of the multi-lumen tubular conduit 500. The ends of the multi-lumen tubular conduit 500 can thus be separated into separate strands (with each strand having one or more conduits) for affixing the devices (detailed above) to each of the conduits at the proximal end (inlet devices) or distal end (outlet devices) of the nasogastric device 100.

With reference now to FIGS. 1 and 2A-2C, the first conduit 202B which serves as the multi-lumen tubular conduit 500 (and encompasses each of the other conduits as well as the inflatable balloon 212) generally comprises an elastomer. The first conduit 202B is used for drug delivery. The first conduit has no perforations and fully covers the inflatable balloon thereby preventing direct contact with esophageal fluids and compressing the balloon to a narrow cross-section.

The first conduit 202B generally comprises an elastomer and encompasses all of the other conduits 204B, 206B and 208B. The first conduit 202B forms a protective cover around all of the other conduits 204B, 206B and 208B. The second, third and fourth conduits (204B, 206B and 208B respectively) may also comprise the same or different elastomer as the first conduit. In an embodiment, the second, third and fourth conduits (204B, 206B and 208B respectively) may comprise a polymer that is not an elastomer.

The elastomer has a glass transition temperature less than 10° C., preferably less than −20° C., and more preferably less than −50° C. Suitable elastomers are biocompatible and non-toxic to living beings. Elastomers that are not inherently compatible may be coated with a layer of a compatible polymer. Elastomers that may be used in the first conduit 202B include polybutadienes, polyisoprenes, styrene-butadiene rubber, poly(styrene)-block-poly(butadiene), poly(acrylonitrile)-block-poly(styrene)-block-poly(butadiene) (ABS), polychloroprenes, epichlorohydrin rubber, polyacrylic rubber, silicone elastomers (polysiloxanes), fluorosilicone elastomers, fluoroelastomers, perfluoroelastomers, polyether block amides (PEBA), chlorosulfonated polyethylene, ethylene propylene diene rubber (EPR), ethylene-vinyl acetate elastomers, or the like, or a combination thereof. Preferred elastomers include polysiloxanes, fluoroelastomers, fluorosilicone elastomers, perfluoroelastomers, or a combination thereof. Preferred elastomers may also include silicone elastomer (possibly coated with hyaluronan, hydrophilic materials, such as sodium hyaluronic salt, or polyurethane), polyvinylidene fluoride, polyamide elastomers, polyurethane elastomers, polyester elastomer, poly(ethylene terephthalate), poly(vinyl chloride), polyethylene, SYLGARD 186, SYLGARD 184, chlorinated neoprene, e.g., CHRONOPRENE or C-PLEX, or a combination thereof.

The other conduits 204B, 206B and 208B may also comprise one or more of the aforementioned elastomers. In an embodiment, the conduits 202B, 204B, 206B and 208B are manufactured from the same elastomer. In another embodiment, the conduits 204B, 206B and 208B are manufactured from a different polymer (or elastomer) from that used in conduit 202B.

In an embodiment, the conduits 204B, 206B and 208B may be manufactured from a polymer that is not elastomeric. These conduits may comprise a thermoplastic or crosslinked polymer that includes a polyacrylic, a polycarbonate, a polyalkyd, a polystyrene, a polyolefin, a polyester, a polyamide, a polyaramid, a polyamideimide, a polyarylate, a polyurethane, an epoxy, a phenolic, a polysiloxane, a polyarylsulfone, a polyethersulfone, a polyphenylene sulfide, a polysulfone, a polyimide, a polyetherimide, a polytetrafluoroethylene, a polyetherketone, a polyether ether ketone, a polyether ketone ketone, a polybenzoxazole, a polyoxadiazole, a polybenzothiazole, a polybenzothiazinophenothiazine, a polypyrazinoquinoxaline, a polypyromellitimide, a polyguinoxaline, a polybenzimidazole, a polyoxindole, a polyoxoisoindoline, a polydioxoisoindoline, a polytriazine, a polypyridazine, a polypiperazine, a polypyridine, a polypiperidine, a polytriazole, a polypyrazole, a polycarborane, a polyoxabicyclononane, a polydibenzofuran, a polyphthalide, a polyacetal, a polyanhydride, a polyvinyl ether, a polyvinyl thioether, a polyvinyl alcohol, a polyvinyl ketone, a polyvinyl halide, a polyvinyl nitrile, a polyvinyl ester, a polysulfonate, a polysulfide, a polythioester, a polysulfone, a polysulfonamide, a polyurea, a polyphosphazene, a polysilazane, a polyolefin, or the like, or a combination thereof.

The inflatable balloon 212 comprises an outer expandable sleeve and an inner inflatable sleeve with sensors disposed therebetween. The balloon is described below.

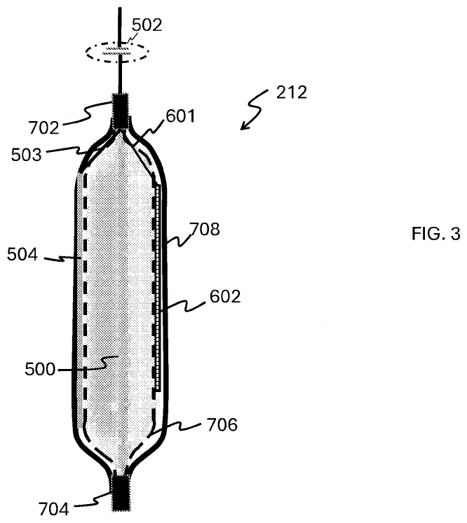

With reference now to the FIGS. 1 and 3, it may be seen that the inflatable balloon 212 comprises two sleeves—an inner inflatable sleeve 706 and an outer elastomeric sleeve 708 that are affixed to an outer surface of the multi-lumen tubular conduit 500 via a first clasp 702 and a second clasp 704. The outer elastomeric sleeve may extend around and up and down the entire device.

The inner inflatable sleeve 706 and outer elastomeric sleeve 708 are both tubular in structure. The inflatable balloon inflates to a diameter larger than that of the multi-lumen tubular conduit 500. The outer elastomeric sleeve has a resting diameter equal to or smaller than that of the inflatable balloon in order to provide compression to the inflatable balloon. Both the inner inflatable sleeve 706 and the outer elastomeric sleeve 708 are expandable and can be expanded upon inflation. The inner inflatable sleeve is made of a material that does not stretch in order to be a dilator. It has narrow ends and then widens in the middle as shown in the drawings and then folds/flattens down in the middle when not in use. They both contract when the fluid pressure is removed. The inflatable balloon is made of a polymer with that has very little capability to stretch such as the polymers shown in Table 1.

Both sleeves are fixed to the outer surface of the conduit by virtue of radio-opaque clasps 702 and 704. When the outer elastomeric sleeve extends along the entire length the clasps are not used. First clasp 702 is closer to the proximal end of the multi-lumen conduit 500 while second clasp 704 is closer to the distal end of the conduit 500. The clasps 702 and 704 can be used to clamp the inner inflatable sleeve 706 and the outer elastomeric sleeve 708 to the multi-lumen conduit 500. Both clasps pinch the ends of the inner inflatable sleeve 706 and the outer elastomeric sleeve 708 to the conduit to create an air-tight contact. As a result, when the inflatable balloon is pressurized, the inner inflatable sleeve 706 and the outer elastomeric sleeve 708 expand. This expansion can be used to prevent strictures and to block the esophagus when desired to provide the esophagus with therapeutic treatment.

The radio-opaque clasps 702 and 704 provide a physician with imaging capabilities. They provide the physician with the ability to locate the inflatable balloon at the appropriate position in the esophagus. The clasps 702 and 704 can comprise a flexible but stiff polymer (at room temperature) that can contain a radio-opaque filler such as barium sulfate, bismuth sub-carbonate, bismuth chloride, tungsten particles, stainless steel bands, platinum/iridium markers, or a combination thereof. The radio-opacity provides the physician to image the nasogastric device (via xrays or computer aided tomography) as it is inserted into the esophagus and to position it accurately to treat injuries and strictures.

With reference now to the FIG. 3, located between the inner inflatable sleeve 706 and the outer elastomeric sleeve 708 are plurality of sensors 504 and/or 506 that provide the physician with information about developing strictures in the esophagus. In an embodiment, one or more a piezoelectric pressure sensors 504 that can detect deformation of the device associated with a newly developed stricture in the esophagus and generates an electronic signal that could provide an alarm and thereby trigger an outpatient visit for balloon dilation. In an embodiment, the piezoelectric sensor 504 that can measure deformation or bending that occurs whenever an esophageal stricture begins to develop. This change in esophageal diameter as detected by the piezoelectric sensor is converted into an electrical signal, which is transmitted via electrical wires 503 and 502 to a microprocessor controlled circuit that activates an alarm light located at the proximal end 104. In an embodiment, the dilation balloon 212 is inflated by a medical personnel in an out-patient department.

There may be one or more piezoelectric sensors 504 located in the space between the inner inflatable tube 706 and the outer elastomeric tube 708. These sensor 504 can extend over a portion of the length of the inflatable balloon 212. In another embodiment, the sensor 504 can extend over the entire length of the inflatable balloon 212. An example of a piezoelectric sensor is polyvinylidene fluoride, barium titanate, lead zirconate titanate, or a combination thereof.

In another embodiment, the nasogastric device 100 may comprise one or more optical fibers 602 that have a surface structure that diffracts light as certain stresses or strains take place on the fiber. The fiber is in communication with a laser source (not shown) via an optical waveguide 601 and receiver sensing device (not shown) located near the proximal end 102. A change in signal as compared to baseline from the strain sensing device, would lead to a light or audible alert. This alert would indicate the necessity of medical intervention such as balloon 212 inflation via the second inlet port to widen the esophagus being narrowed by stricture formation. It is to be noted that the opposite end of the optical fiber or the piezo electric sensors are located at the proximal end of the nasogastric device 100.

Both the inner inflatable sleeve 706 and the outer elastomeric sleeve 708 can use the elastomers listed above. The use of an elastomer in the inner inflatable sleeve 706 and/or the outer elastomeric sleeve 708 facilitates a return of the inflatable balloon to its original non-inflated state when inflation is no longer desired. As noted above, the balloon is inflated when a stricture develops in the esophagus. The balloon is inflated to counter the stricture. When the stricture abates (as a result of outward pressure exerted by the balloon), the pressure on the balloon is removed and the elastomeric sleeve returns to its original state because of its elastic properties. The reduction of the balloon to its original shape facilitates food and other fluids to bypass the nasogastric device and to continue with normal digestive processes. In other words, the expansion of the balloon causes the elastomer to go from a low energy, high entropy state (prior to expansion) to a high energy, low entropy state. When the pressure on the elastomer is removed, it returns to its low energy state deflating the balloon in the process and permitting normal digestive processes to continue. Feeding through the conduit is possible even when the balloon is inflated due to the feeding tube conduit.

In summary, the balloon 212 comprises an inner inflatable sleeve 706 that is operative to receive a fluid via outlet port 204C. The balloon 212 further comprises an outer expandable sleeve 708 that surrounds in the inner inflatable sleeve 706 and that comprises an elastomer. Both the inner sleeve 706 and the outer expandable sleeve 708 are disposed on the outside of the multi-lumen conduit 500 and are held in position by clasps 702 and 704. The respective clasps pin the inner inflatable sleeve 706 and the outer sleeve 708 to the conduit 500 to form an airtight enclosure (the inflatable balloon 212) that can be inflated when desired. The elastomer provides a counterbalancing force that restores the balloon 212 to its folded original size. The inner inflatable sleeve 706 may also provide a counterbalancing force that restores the balloon 212 to its original size.

The mechanical properties of the balloon can be tailored by varying one or more reactants in the material. The Young's modulus may vary from 10 Pascals (Pa) to 1 Gigapascal (GPa). The initial radius of the collapsed balloon ranges from about 2 millimeters to about 4 centimeters; and this radius may reach up to about 1000% its initial value when the balloon is inflated. In exemplary aspects, the initial length of the balloon may vary from about 0.5 to 30 centimeters. In certain aspects, the balloon material is designed to be stable against degradation in vivo for a period of at least one year. The balloon may be sterilized using techniques appropriate for medical tubing, including autoclaving, ethylene oxide treatments, plasma treatments, and irradiation. The balloon may contain tracer molecules (radiopaque markers, near infrared dyes, fluoroscopic markers, or other agent(s)) to allow visualization of the placement of the device.

In further aspects, the radius of the balloon 212 after it is inflated is between about 10 millimeters and 40 millimeters, depending on pediatric or adult patient requirements. For example, the radius of the balloon 212 after it is inflated is between about 10 mm-35 mm, about 10 mm-30 mm, about 10 mm-25 mm, about 10 mm-20 mm, about 10 mm-15 mm, about 15 mm-40 mm, about 20 mm-40 mm, about 25 mm-40 mm, about 30 mm-40 mm, or about 35 mm-40 mm, depending on pediatric or adult patient requirements. In additional exemplary aspects, the radius of the balloon 212 when collapsed and not inflated may be between about 2 and 10 millimeters in diameter. For example, the radius of the balloon when collapsed and not inflated may be between about 2 and 9 mm, about 2 and 8 mm, about 2 and 7 mm, about 2 and 6 mm, about 2 and 5 mm, or about 2 and 3 mm in diameter. The balloon 212 will remain deflated until a time is needed to deliver drugs or expand a potential stricture. In further aspects, the radius of the deflated balloon and conduits should be less than the current diameter of the esophagus as to prevent the native esophagus from continuously touching the balloon and device.

The average distance between the inlet ports and the inflatable balloon 212 may be 8 to 25 centimeters, preferably 9 to 20 centimeters, and more preferably 10 to 15 centimeters. The balloon 212 may have a length of 2 to 5 centimeters, preferably 3 to 4 centimeters. The distance between the distal end of the balloon and the end of the outlet port 208C may be 8 to 18 centimeters, preferably 9 to 15 centimeters.

The Table below shows some of the exemplary materials that may be used for the different parts of the nasogastric device.

TABLE

| | |
|---|---|
| Multi-lumen | poly(vinyl chloride) (PVC), urethanes, silicone, |
| Conduit | perfluoroelastomers, polytetrafluoroethylene |
| Inner inflatable | Thermoplastic or thermoplastic elastomer, for |
| sleeve (dilation | example thermoplastic PEBAX (a block |
| balloon) | copolymer variation of PEBA (polyether block |
| | amide) that is made up of polyamide and polyether |
| | backbone blocks) or polyethylene terephthalate |
| | (PET), thermoplastic polyurethane (e.g., |
| | PELLATHANE) |
| Elastomeric cover | Silicone elastomer (possibly coated with |
| (elastomeric sleeve) | hyaluronan, hydrophilic materials, such as sodium |
| | hyaluronic salt, or polyurethane), polyvinylidene |
| | fluoride, polyamide elastomers, polyurethane |
| | elastomers, polyester elastomer, poly(ethylene |
| | terephthalate), poly(vinyl chloride), polyethylene, |
| | SYLGARD 186, SYLGARD 184, chlorinated |
| | neoprene, e.g., CHRONOPRENE or C-PLEX |
| Stricture sensor | Piezoelectric flexible polymers such as |
| | polyvinylprolidone (PVP) polyvinylidene fluoride |
| | (PVDF), flexible polymers containing zirconate |
| | titanate (PZT) nanoparticles or nylon in the form |
| | of a film or fiber |
| | Optical fiber - a core of silica, PMMA (acrylic) |
| | or polycarbonate |
| | Measured by a difference in volume of the initial |
| | dilation balloon volume vs strictured dilation |
| | balloon volume |
| Radio-opaque | Stainless steel band, platinum/iridium markers |
| Markers | |
| Tubing Connections | Polypropylene |
| (Leuer Ports) | |
| Main Feeding | poly(vinyl chloride) (PVC), urethanes, silicone |
| Conduit | |
| Balloon Inflation | poly(vinyl chloride) (PVC), PEBAX, |
| Conduit | PELLATHANE, polyurethanes, silicone. |

### Outlet Ports

The multi-lumen tubular conduit 500 contains a plurality of outlet ports—the first outlet port 202C, the second outlet port 204C, the third outlet port 206C and the fourth outlet port 208C (located at the distal end 104) that permit materials that are introduced into the inlet ports (at the proximal end 102) to exit the nasogastric device 100. All ports are located downstream of the proximal end 102 of the nasogastric device 100 and one of the outlet ports open into the inner inflatable sleeve 706 of inflatable balloon 212 (see FIG. 1).

Each outlet port may be located at an extension that protrudes from the particular conduit in the multi-lumen tubular conduit 500. For example, the first outlet port 202C is located at an extension that protrudes from first conduit 202B (located in the multi-lumen tubular conduit 500). In another embodiment, the outlet port may be a cannula (not shown) that pierces the conduit and serves as an outlet port from which the contents of the conduit may be extracted.

The first outlet port 202C lies upstream of the inflatable balloon 212. It is used to deliver therapeutic materials (biologically active agents) to the region around the stricture. Suitable therapeutic materials include anti-proliferative/antimitotic agents including natural products such as vinca alkaloids (e.g., vinblastine, vincristine, and vinorelbine), paclitaxel, epidipodophyllotoxins (e.g., etoposide, teniposide), antibiotics (e.g., dactinomycin, actinomycin D, daunorubicin, doxorubicin, penicillin V, penicillin G, ampicillin, amoxicillin, cephalosporin, tetracycline, doxycycline, minocycline, demeclocycline, erythromycin, aminoglycoside antibiotics, polypeptide antibiotics, nystatin, griseofulvin, and idarubicin), anthracyclines, mitoxantrone, bleomycins, plicamycin, mithramycin and mitomycin, enzymes (L-asparaginase, which systemically metabolizes L-asparagine and deprives cells which do not have the capacity to synthesize their own asparagine), antiplatelet agents such as G(GP) IIb/IIIa inhibitors and vitronectin receptor antagonists, anti-proliferative/antimitotic alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC), anti-proliferative/antimitotic antimetabolites such as folic acid analogs (e.g., methotrexate), pyrimidine analogs (e.g., fluorouracil, floxuridine, cytarabine), purine analogs and related inhibitors (e.g., mercaptopurine, thioguanine, pentostatin and 2-chlorodeoxyadenosine {cladribine}), platinum coordination complexes (e.g., cisplatin, carboplatin), procarbazine, hydroxyurea, mitotane, aminoglutethimide, hormones (e.g., estrogen), anti-coagulants (e.g., heparin, synthetic heparin salts and other inhibitors of thrombin), fibrinolytic agents (e.g., tissue plasminogen activator, streptokinase and urokinase), aspirin, dipyridamole, ticlopidine, clopidogrel, abciximab, antimigratory, antisecretory (e.g., breveldin), anti-inflammatory: such as adrenocortical steroids (e.g., cortisol, cortisone, fludrocortisone, prednisone, prednisolone, 6α-methylprednisolone, triamcinolone, betamethasone, and dexamethasone), non-steroidal agents (e.g., salicylic acid derivatives such as aspirin, para-aminophenol derivatives such as acetominophen, indole and indene acetic acids (e.g., indomethacin, sulindac, etodalac), heteroaryl acetic acids (e.g., tolmetin, diclofenac, ketorolac), arylpropionic acids (e.g., ibuprofen and derivatives), anthranilic acids (e.g., mefenamic acid, meclofenamic acid), enolic acids (e.g., piroxicam, tenoxicam, phenylbutazone, oxyphenthatrazone), nabumetone, gold compounds (e.g., auranofin, aurothioglucose, gold sodium thiomalate), immunosuppressives (e.g., cyclosporine, tacrolimus (FK-506), sirolimus (e.g., rapamycin, azathioprine, mycophenolate mofetil), angiogenic agents such as vascular endothelial growth factor (VEGF), fibroblast growth factor (FGF), angiotensin receptor blockers, nitric oxide donors, anti-sense oligionucleotides and combinations thereof, cell cycle inhibitors, mTOR inhibitors, and growth factor receptor signal transduction kinase inhibitors, retenoids, cyclin/CDK inhibitors, HMG co-enzyme reductase inhibitors (statins) or protease inhibitors.

The second outlet port (204C) lies inside the inner inflatable sleeve 706 of the inflatable balloon 212 and is in fluid communication with the second inlet port 204B via second conduit 204B. The second outlet port 204C protrudes from the multi-lumen conduit 500 as traverses the length of the inflatable balloon 212. The multi-lumen conduit 500 extends through the balloon 500 and emerges at the distal end of the inflatable balloon where it extends to provide third outlet port 206C and fourth outlet port 208C.

The second outlet port is operative to transmit a fluid that expands an inflatable balloon 212 so that it can be used to expand strictures within the esophagus. The inner inflatable sleeve 706 receives a fluid via outlet port 204C (which receives the fluid directly from an inlet port 204A) to cause the balloon to inflate and expand any strictures in the esophagus. In an embodiment, the fluid used to expand the balloon 212 is a liquid. In an embodiment, the liquid is water.

The third outlet port 206C may be used for aspirating the region downstream of the inflatable balloon. It protrudes from the multi-lumen conduit 500 via an extension. As noted above, the multi-lumen conduit 500 The third outlet port may therefore be in fluid communication with a pump at the third inlet port 206A, which facilitates removal of excess biological agents. In an embodiment, the third outlet port 206C may be fitted with a device such as a tweezer or pincer (not shown), which may be activated to remove a tissue sample for a biopsy. The tweezer or pincer may also be radio-opaque so that it can be imaged during a surgical operation.

The fourth outlet port 208C is the feeding tube outlet port and it is the distal-most port that extends from the multi-lumen conduit 500 (See FIG. 1). It lies downstream of all of the outlet ports 202C, 204C and 206C. It is in communication with the fourth inlet port 208A and the fourth conduit 208B. The feeding tube outlet port is used to feed the patient with nutrition in the event that the inflatable balloon has to remain in position in its expanded state for an extended period of time.

In an embodiment, at least a portion of the feeding tube includes perforations (not shown) configured to allow material injected into said tube to exit freely into the stomach. In an embodiment, said perforations have a length of between 1 and 8 millimeters. In an embodiment, at least the last 5 centimeters of the distal end of the feeding tube includes such perforations. In an embodiment, the distal end of the feeding tube extends between about 1 and 10 centimeters below the distal end of the inflatable balloon.

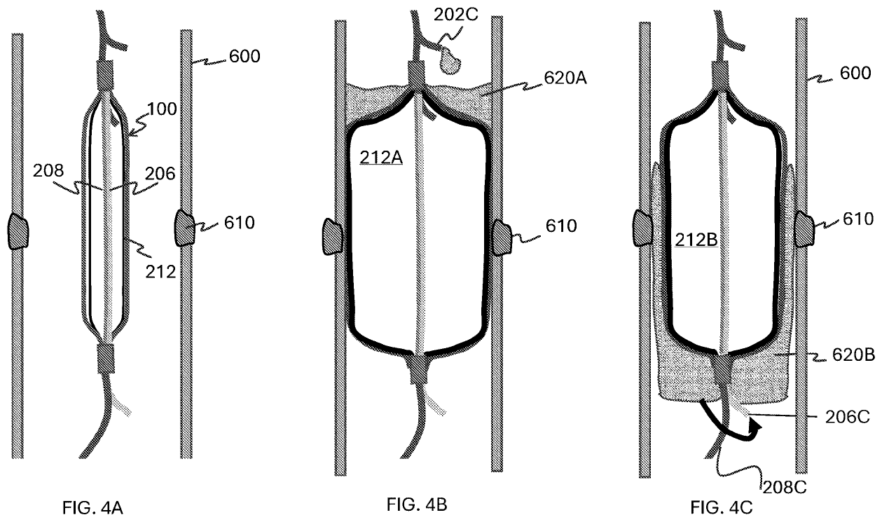

FIGS. 4A, 4B and 4C depict one exemplary embodiment of using the nasogastric device 100 in the esophagus 600 at a location where an injury may cause a stricture 610 to appear. In the FIG. 4A, the inflatable balloon 212 is not expanded. Upon noting the development of a stricture 610, the balloon may be inflated using a pump at the second inlet port 204A as seen in the FIG. 4B. When the outer surface of the sleeve contacts the inner surfaces of the esophagus (because the balloon is in its fully inflated state 212A), a biological agent that mitigates the stricture may be released from the first outlet port 202C. The contact between the outer surface of the inflatable balloon 212 and the inner surface of the esophagus prevents the biologically active agent from travelling downwards. In short the inflatable balloon may be inflated to a state 212A where it can act as a valve to prevent the movement of the biologically active agent away from the site of the stricture 610 until so desired.

After appropriate duration of contact time of the biologically active agent has been reached, the inflatable balloon 212 is deflated and the balloon returns to a state 212B where it leaves a space between its outer surface and the inner surface of the esophagus. This annular space between the respective surfaces of the balloon and the esophagus permits the biologically active agent to move towards the third outlet valve 206C, where it can be aspirated and removed from the esophagus. The process depicted in the FIGS. 4A, 4B and 4C may be repeated until the stricture is completely reduced. The nasogastric device may be left in the esophagus until the problem has abated. When desired the nasogastric device may be removed from the body via the nasal passage and stored for further use. FIG. 4B shows the biologically active agent 620A above the inflatable balloon 212 and FIG. 4C shows the biologically active agent 620B after it is released to move downstream of the balloon.

In an embodiment, at least a portion of the distal end includes perforations that have a length of between 1 and 3 millimeters. In an embodiment, the inflatable balloon has an initial radius of between about 5 millimeters and 4 centimeters. In an embodiment, the inflatable balloon has an initial length of between about 0.5 and 30 centimeters. In an embodiment, the elastomeric sleeve fully surrounds said inflatable balloon. In an embodiment, the device further includes at least one radiopaque marker provided near a distal portion of an element of said nasogastric device. In an embodiment, the device further includes ports configured at the proximal ends of each of said four tubes. In an embodiment, the elastomeric sleeve may be textured to improve stenting function. In an embodiment, the balloon/elastomeric sleeve configuration provides a structure that stents the area, but that can also be dilated by using pneumatic pressure to expand the balloon.

In summary, temporary expansion of the inflation balloon, coinciding with delivery of therapeutic substances by the injectable conduit, seals off fluid transport down the esophagus thereby promoting accumulation and full circumferential exposure of the esophagus to the therapeutic for an extended time period. Upon deflation of the balloon the accumulated liquid therapeutic will further coat the esophagus or may be aspirated up by the aspiration tube to reduce or avoid non-localized dosing of the therapeutic. Liquids, shear thinning fluids, or gels may also be injected through the injectable conduit when the device is placed to break down therapeutic vehicles.

In one embodiment, in one manner of producing the device, a suitable polymer (such as for example an elastomer) is subjected to crosshead co-extrusion using 2 to 4 mandrils to produce the multi-lumen, multi-tubular conduit used in the nasogastric device of the FIGS. 2A-2C. The mandrels may then be removed after the curing or hardening of the polymer. When the polymer is a thermoset, it may be cured by adding to it an initiator and crosslinker. When the polymer is a thermoplastic, it may be hardened by cooling it.

Following the manufacturing of the multi-tubular conduit, inlet ports may be drilled into the respective conduits. These inlet ports may be fitted with inlet devices such as injection pumps, squeeze pumps, suction pumps, and so on. The respective outlet ports 206A and 206C may then protrude through holes in the first conduit 202B (which enclosed all of the other conduits). The device may then be packaged and stored for shipping.

While the invention has been described with reference to some embodiments, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiments disclosed as the best mode contemplated for carrying out this invention, but that the invention will include all embodiments falling within the scope of the appended claims.

1. A nasogastric device comprising:
a multi-lumen tubular conduit having a proximal end and a distal end with a plurality of conduits arranged therebetween;
a plurality of inlet ports located at the proximal end in fluid communication with the plurality of conduits; where the plurality of conduits are arranged such that at least one conduit of the plurality of conduits is located with a wall of another conduit;
where at least one conduit of the plurality of conduits is in fluid communication with an inflatable balloon; and
where at least one conduit of the plurality of conduits is in fluid communication with a drug delivery inlet port.

2. The nasogastric device of claim 1, wherein at least three conduits are located within the wall of the another conduit.

3. The nasogastric device of claim 1, wherein the another conduit comprises an elastic outer sleeve that envelops at least a portion of the three conduits.

4. The nasogastric device of claim 4, wherein the three conduits include a drug delivery conduit, a conduit that is operative to aspirate a region downstream of the inflatable balloon and a feed tube conduit.

5. The nasogastric device of claim 1, wherein the inflatable balloon comprises one or more piezoelectric pressure sensor that is operative to detect a change in pressure around the inflatable balloon.

6. The nasogastric device of claim 1, wherein the nasogastric device includes an optical fiber with a strain sensing surface in communication with a laser source and receiver that is operative to sense the stress or strain due to the stricture forming.

7. The nasogastric device of claim 1, where the inflatable balloon comprises an outer elastomeric sleeve with an inner inflatable sleeve disposed therein; where the inner inflatable sleeve is operative to expand in response to pressurized fluid while the outer elastomeric sleeve is operative to enable the inflatable balloon to return to its original size.

8. A nasogastric device, the device comprising:
a multi-lumen tubular conduit having a proximal end and a distal end with a plurality of conduits arranged therebetween; the proximal end having a plurality of inlet ports and the distal end having a plurality of outlet ports;
where a first conduit of the plurality of conduits is in communication with a first inlet port and a first outlet port; where the first inlet port is operative deliver a biologically active agent to the first outlet port; and
where a second conduit of the plurality of conduits is in communication with a second inlet port and a second outlet port; where the second inlet port is operative to pressurize an inflatable balloon that lies closer to the distal end that the first outlet port; where the second conduit lies inside the first conduit or lies adjacent to the first conduit.

9. The nasogastric device of claim 8, where the plurality of conduits further comprises a third conduit; where the third conduit is in fluid communication with a third inlet port and a third outlet port; and where the third outlet port is operative to aspirate a region surrounding the third outlet port or to remove a tissue from a region surrounding the third outlet port for a biopsy.

10. The nasogastric device of claim 9, where the third outlet port lies downstream of the inflatable balloon.

11. The nasogastric device of claim 9, where the plurality of conduits further comprises a fourth conduit; where the fourth conduit is in fluid communication with a fourth inlet port and a third fourth outlet port; and where the fourth outlet port is located downstream of the inflatable balloon where the fourth outlet port is operative to feed a region of the body with nutrients after the balloon is inflated.

12. The nasogastric device of claim 8, where the inflatable balloon comprises an elastomeric outer sleeve and an inner sleeve disposed within the outer sleeve, where the inner sleeve is operative to expand in response to a pressurized fluid while the elastomeric outer sleeve is operative to enable the inflatable balloon to return to its original size.

13. The nasogastric device of claim 8, where the inflatable balloon comprises a piezoelectric pressure sensor that is operative to detect a stricture and/or wherein the inflatable balloon comprises an optical fiber sensor in optical communication with a laser and receiving device that is operative to detect a stricture.

14. The nasogastric device of claim 8, where a third conduit and/or a fourth conduit lie within the first conduit.

15. The nasogastric device of claim 8, where a third conduit and/or a fourth conduit lie adjacent to the first conduit.

16. The nasogastric device of claim 8, where the multi-lumen tubular conduit is the first conduit.

17. A method of treating an esophageal stricture with a nasogastric device, the method comprising:
threading the nasogastric device through a nose of a living being into an esophagus; and
inflating an inflatable balloon in the nasogastric device to expand a stricture in the esophagus;
where the nasogastric device comprises:
a multi-lumen tubular conduit having a proximal end and a distal end with a plurality of conduits arranged therebetween; the proximal end having a plurality of inlet ports and the distal end having a plurality of outlet ports;
where a first conduit of the plurality of conduits is in communication with a first inlet port and a first outlet port; where the first inlet port is operative deliver a biologically active agent to the first outlet port; and
where a second conduit of the plurality of conduits is in communication with a second inlet port and a second outlet port; where the second inlet port is operative to pressurize the inflatable balloon that lies closer to the distal end that the first outlet port; where the second conduit lies inside the first conduit or lies adjacent to the first conduit.

18. The method of claim 17, further comprising transporting a biologically active agent through the first conduit to prevent a stricture.

19. The method of claim 18, further comprising aspirating a region downstream of the inflatable balloon after the balloon is inflated.

20. The method of claim 19, further comprising transporting nutrients to a location downstream of the inflatable balloon after the balloon is inflated.
